(19) United States
(12) Patent Application Publication
(10) Pub. No.: US 2024/0299461 A1
(43) Pub. Date: Sep. 12, 2024

(54) CELL-FREE FAT EXTRACT FOR TREATING OVARIAN INSUFFICIENCY

(71) Applicant: SHANGHAI SEME CELL TECHNOLOGY CO., LTD., Shanghai (CN)

(72) Inventor: Wenjie ZHANG, Shanghai (CN)

(21) Appl. No.: 18/578,698

(22) PCT Filed: Jun. 13, 2022
(86) PCT No.: PCT/CN2022/098413

§ 371 Date: Jan. 11, 2024

(30) Foreign Application Priority Data
Jul. 14, 2021 (CN) 202110795666.0

(51) Int. Cl. A61K 35/35; A61P 5/08; A61P 5/30; A61P 15/08

(52) U.S. Cl. CPC A61K 35/35; A61P 5/08; A61P 5/30; A61P 15/08

(57) ABSTRACT

Disclosed is the use of a cell-free fat extract for preparing a composition or a preparation. The composition or the preparation is used for preventing and/or treating ovarian insufficiency and/or complications thereof. The cell-free fat extract has an excellent therapeutic effect on ovarian insufficiency and/or complications thereof.

### FIELD OF THE INVENTION

The present invention relates to the field of pharmaceuticals, specifically, relates to the use of cell-free fat extract for treating ovarian insufficiency.

### BACKGROUND OF THE INVENTION

Premature ovarian insufficiency (POI), also known as “hypoovarianism”, refers to acquired high gonadotropic dysplasia before the age of 40. The progression of women from the fetus to old age is a gradual physiological process, and the ovary, as a gonadal organs, performs important physiological functions of cyclical ovulation and secretion of sex hormones. With the advances of chemotherapy regimens, many patients with malignant tumor have been able to survive for a long time or even be cured. However, due to chemotherapy induced ovarian dysfunction, premature amenorrhea and infertility occur, which affects the life quality of female patients. To preserve fertility and physiological functions while treating diseases is a problem that female patients with malignant tumors are eager to solve. At present, hormone replacement therapy is mainly used at home and abroad, but long-term use has led to an increase in the incidence of ovarian cancer, resulting in significant controversy over application of the therapy. Therefore, there is no reliable treatment method that can help restore female ovarian function yet. Therefore, it is necessary in this field to develop a drug that can effectively treat ovarian insufficiency.

### SUMMARY OF THE INVENTION

The purpose of the present invention is to provide a use of cell-free fat extract in the prevention and/or treatment of ovarian insufficiency and/or complications thereof.

A first aspect of the present invention provides a use of a cell-free fat extract for preparing a composition or a preparation, the composition or the preparation is used for preventing and/or treating ovarian insufficiency and/or complications thereof.

In another preferred embodiment, a subject with ovarian insufficiency and/or complications thereof is human or non-human mammal.

In another preferred embodiment, the complications of ovarian insufficiency are selected from the group consisting of secondary amenorrhea, decreased estrogen level, increased gonadotropin level, decreased number of follicles, decreased follicle quality, premature amenorrhea, infertility, and the combinations thereof.

In another preferred embodiment, the ovarian insufficiency includes ovarian insufficiency caused by chemical damage.

In another preferred embodiment, the chemical damage includes damage caused by chemotherapeutic drugs.

In another preferred embodiment, the chemotherapeutic drugs comprise chemotherapeutic drugs for preventing and/or treating tumor.

In another preferred embodiment, the chemotherapeutic drugs include paclitaxel or doxorubicin.

In another preferred embodiment, the chemotherapeutic drugs are selected from the group consisting of cyclophosphamide, busulfan, paclitaxel, doxorubicin, and the combinations thereof.

In another preferred embodiment, the preventing and/or treating ovarian insufficiency and/or complications thereof include:
(i) promoting the recovery of ovarian function;
(ii) enhancing fertility.

In another preferred embodiment, the promoting the recovery of ovarian function includes one or more features selected from the group consisting of:
(i) increasing estradiol hormone level;
(ii) increasing anti-Müllerian hormone level;
(iii) decreasing follicle stimulating hormone level;
(iv) restoring follicular structure.

In another preferred embodiment, the enhancing fertility includes increasing the number of births.

In another preferred embodiment, the cell-free fat extract is a cell-free fat extract extracted from fat from human or non-human mammals.

In another preferred embodiment, the non-human mammals are monkeys, chimpanzees, cows, pigs, dogs, sheep, mice, or rabbits.

In another preferred embodiment, the composition or the preparation comprises a pharmaceutical composition or preparation, a food composition or preparation, a health product composition or preparation, or a dietary supplement.

In another preferred embodiment, the composition or the preparation further comprises pharmaceutically, food, health product, or dietary acceptable carriers.

In another preferred embodiment, the composition or the preparation further comprises other drugs for preventing and/or treating ovarian insufficiency and/or complications thereof.

In another preferred embodiment, other drugs for preventing and/or treating ovarian insufficiency and/or complications thereof are selected from the group consisting of hormone replacement therapy (HRT), dehydroepiandrosterone (DHEA), clomiphene, etc.

In another preferred embodiment, the dosage form of the composition or the preparation is an oral preparation, a topical preparation, or an injectable preparation.

In another preferred embodiment, the injection formulation is intravenous injection or intramuscular injection.

In another preferred embodiment, the dosage form of the composition or the preparation is a solid dosage form, a semi-solid dosage form, or a liquid dosage form, such as solution, gel, cream, emulsion, ointment, cream, paste, cake, powder, patch, etc.

In another preferred embodiment, the dosage form of the composition or the preparation is powder, granule, capsule, injection, tincture, oral liquid, tablet or lozenge.

In another preferred embodiment, the composition or the preparation is administered externally, topically, or by injection.

In another preferred embodiment, the cell-free fat extract contains no cells and contains no lipid droplets.

In another preferred embodiment, the lipid droplets are oil droplets released after the fat cells are disrupted.

In another preferred embodiment, “contains no lipid droplets” means that in the cell-free fat extract, the volume of oil droplets accounts for less than 1% of the total liquid, preferably less than 0.5%, more preferably less than 0.1%.

In another preferred embodiment, the cells are selected from the group consisting of endothelial cells, adipose stem cells, macrophages, and stromal cells.

In another preferred embodiment, the “cell-free” means that the average number of cells in 1 ml of the cell-free fat extract is ≤1, preferably ≤0.5, more preferably ≤0.1, or 0.

In another preferred embodiment, the cell-free fat extract is a naturally obtained nanofat extract without additive.

In another preferred embodiment, “without additive” means that no solutions, solvents, small molecules, chemical agents, and biological additives are added during the preparation of the cell-free fat extract except rinsing step.

In another preferred embodiment, the cell-free fat extract is obtained by subjecting an fatty tissue to emulsification and then centrifugation.

In another preferred embodiment, the cell-free fat extract comprises one or more components selected from the groups consisting of IGF-1, BDNF, GDNF, TGF-β1, HGF, bFGF, VEGF, TGF-β1, PDGF, EGF, NT-3, GH, G-CSF, and the combinations thereof.

In another preferred embodiment, the cell-free fat extract contains but is not limited to one or more components selected from the groups consisting of IGF-1, BDNF, GDNF, bFGF, VEGF, TGF-β1, HGF, PDGF, and the combinations thereof.

In another preferred embodiment, the cell-free fat extract is a cell-free fat extract liquid. In another preferred embodiment, in the cell-free fat extract, the concentration of IGF-1 is 5000-3000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, and more preferably 170-190 pg/ml.

In another preferred embodiment, the weight ratio of IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, preferably 6-12:1, and most preferably 8-9.5:1.

In another preferred embodiment, the weight ratio of GDNF to VEGF is 2-20:1, preferably 4-15:1, preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β1 to VEGF is 1-20:1, preferably 1-15:1, more preferably 1-10:1, more preferably 2-8:1, and more preferably 4-6:1.

In another preferred embodiment, the weight ratio of HGF to VEGF is 1-20:1, preferably 1-15:1, more preferably 1-10:1, more preferably 2-8:1, and more preferably 4-55:1.

In another preferred embodiment, the weight ratio of PDGF to VEGF is 0.1-3:1, preferably 0.2-2:1, more preferably 0.4-1.5:1, and most preferably 0.7-1.2:1.

In another preferred embodiment, the cell-free fat extract is prepared by the following method:
(1) providing an fatty tissue raw material, shredding the fatty tissue raw material, and rinsing (eg, with physiological saline), thereby obtaining a rinsed fatty tissue;
(2) centrifuging the rinsed fatty tissue to obtain a layered mixture;
(3) removing the oil layer on top and the water layer at the bottom from the layered mixture and collecting the intermediate layer (that is, the fat layer containing fat cells);
(4) subjecting the intermediate layer to emulsification to obtain a emulsified fat mixture (also called nano-fat);
(5) subjecting the emulsified fat mixture to centrifugation to obtain a intermediate liquid layer, which is a fat primary extract; and
(6) subjecting the fat primary extract to filtration and sterilization to obtain the cell-free fat extract.

The second aspect of the present invention provides a method for preparing a cell-free fat extract, comprising the steps:
(1) providing an fatty tissue raw material, shredding the fatty tissue raw material, and rinsing (eg, with physiological saline), thereby obtaining a rinsed fatty tissue;
(2) centrifuging the rinsed fatty tissue to obtain a layered mixture;
(3) removing the oil layer on top and the water layer at the bottom from the layered mixture and collecting the intermediate layer (that is, the fat layer containing fat cells);
(4) subjecting the intermediate layer to emulsification to obtain a emulsified fat mixture (also called nano-fat);
(5) subjecting the emulsified fat mixture to centrifugation to obtain a intermediate liquid layer, which is a fat primary extract; and
(6) subjecting the fat primary extract to filtration and sterilization to obtain the cell-free fat extract.

In another preferred embodiment, the cell-free fat extract is as described in the first aspect of the present invention.

In another preferred embodiment, in step (2), the centrifugation is performed at 800-2500 g, preferably 800-2000 g, more preferably 1000-1500 g, and most preferably 1100-1300 g.

In another preferred embodiment, in step (2), the centrifugation time is 1-15 minutes, preferably 1-10 minutes, more preferably 1-8 minutes, and most preferably 1-5 minutes.

In another preferred embodiment, the centrifugation temperature is 2-6° C.

In another preferred embodiment, in step (4), the emulsification is mechanical emulsification.

In another preferred embodiment, the mechanical emulsification is performed by repeatedly blowing through a syringe for many times (e.g. 20-200 times, preferably 20-150 times, preferably 20-100 times, preferably 30-50 times).

In another preferred embodiment, the blowing method is to repeatedly and uniformly push back through two 10 ml injection syringes connected to a three-way tube.

In another preferred embodiment, in step (4), the emulsification is a method of breaking up by a tissue homogenizer.

In another preferred embodiment, step (5) further comprises subjecting the emulsified fat mixture to freezing and thawing treatment before the emulsified fat mixture is subjected to centrifugation.

In another preferred embodiment, after freezing and thawing treatment, the thawed mixture is used for centrifugation.

In another preferred embodiment, the freezing temperature is −50° C. to −120° C., preferably −60° C. to −100° C., and more preferably −70° C. to −90° C.

In another preferred embodiment, the thawing temperature is 20-40° C., preferably 25-40° C., and more preferably 37° C.

In another preferred embodiment, the number of cycles for freezing and thawing is 1-5 (preferably 1, 2, 3, or 4).

In another preferred embodiment, in step (5), after centrifugation, the emulsified fat mixture is layered into four layers: the first layer is an oil layer, the second layer is a residual fat tissue layer, the third layer is a liquid layer (i.e. the intermediate liquid layer), and the fourth layer is a cell/tissue debris precipitation layer.

In another preferred embodiment, in step (5), the centrifugation is performed at 800-2500 g, preferably 800-2000 g, more preferably 1000-1500 g, and most preferably 1100-1300 g.

In another preferred embodiment, in step (5), the centrifugation time is 1-15 minutes, preferably 1-10 minutes, more preferably 2-8 minutes, and most preferably 3-7 minutes. In another preferred embodiment, the centrifugation temperature is 2-6° C.

In another preferred embodiment, in step (5), the first layer, the second layer, the third layer and the fourth layer are arranged sequentially from top to bottom.

In another preferred embodiment, in step (5), the intermediate liquid layer is a transparent or substantially transparent layer.

In another preferred embodiment, in step (6), the filtration is capable of removing fat cells from the fat primary extract.

In another preferred embodiment, in step (6), the filtration and sterilization are carried out through a filter (such as a 0.22 μm microporous filter membrane).

In another preferred embodiment, the filter is a microporous membrane filter.

In another preferred embodiment, the pore size of the microporous filter membrane is 0.05-0.8 μm, preferably 0.1-0.5 μm, more preferably 0.1-0.4 μm, more preferably 0.15-0.3 μm, more preferably 0.2-0.25 μm, and most preferably 0.22 μm.

In another preferred embodiment, in step (6), the filtration and sterilization are carried out by first filtering through a first filter that can filter cells, and then through a second filter (such as a 0.22 μm filter) that can filter pathogens (such as bacteria).

In another preferred embodiment, step (6) further comprises subpackaging the fat extract to form a subpackaged product. (The extract after subpackaging may be stored at −20° C. for later use; may be thawed at low temperature (e.g. −4° C.) or at room temperature then used directly, or thawed and stored at a low temperature (e.g. 4° C.) for a period of time before use).

The third aspect of the present invention provides a cell-free fat extract, the cell-free fat extract is obtained by the method described in the second aspect of the present invention.

The fourth aspect of the present invention provides a composition or preparation, comprising (a) a cell-free fat extract as described in the third aspect of the present invention; and (b) pharmaceutically, food, health care product or dietary acceptable carriers or excipients.

In another preferred embodiment, the composition is a pharmaceutical composition, a food composition, a health care composition or a dietary supplement.

In another preferred embodiment, the dosage form of the composition or the preparation is an oral preparation, a topical preparation or an injection preparation.

In another preferred embodiment, the dosage form of the composition or the preparation is powder, granule, capsule, injection, tincture, oral liquid, tablet or lozenge.

In another preferred embodiment, the injection is an intravenous or intramuscular injection.

In another preferred embodiment, the dosage form of the composition or the preparation is a solid dosage form, a semi-solid dosage form, or a liquid dosage form, such as solution, gel, cream, emulsion, ointment, cream, paste, cake, powder, patch, etc.

In another preferred embodiment, in the composition or the preparation, the mass percentage of the cell-free fat extract is 5 wt %, preferably 1-20 wt %, based on the total weight of the composition or the preparation.

The fifth aspect of the present invention provides a method for preparing the composition or the preparation according to the fourth aspect of the present invention, comprising the step of: mixing the cell-free fat extract according to the third aspect of the present invention with pharmaceutically, food, health care product or dietary acceptable carriers or excipients to form the composition or the preparation.

The sixth aspect of the present invention provides a method for preventing and/or treating ovarian insufficiency and/or complications thereof, comprising administering the cell-free fat extract according to the third aspect of the invention to a subject in need thereof.

In another preferred embodiment, the subject is a human or a non-human mammal.

In another preferred embodiment, the non-human mammal comprises a rodent, such as a rat, a mouse.

In another preferred embodiment, the mode of administration is oral, topical, or injection administration.

It should be understood that within the scope of the present invention, the above-described technical features of the present invention and the technical features described in detail below (e.g., examples) may be combined with each other to constitute a new or preferred technical solution. Limited by space, it will not be repeated herein.

### DESCRIPTION OF THE DRAWINGS

FIG. 1 shows the effect of CEFFE on E2 hormone levels in POI mice.

FIG. 2 shows the effect of CEFFE on AMH hormone levels in POI mice.

FIG. 3 shows the effect of CEFFE on FSH hormone levels in POI mice.

FIG. 4 shows the ovarian histological examination results of POI mice before and after CEFFE treatment. FIG. 4A: POI for 2 weeks; FIG. 4B: POI+CF treatment for 2 weeks; FIG. 4C: POI for 4 weeks; FIG. 4D: POI+CF treatment for 4 weeks; FIG. 4E: Normal ovaries.

### DETAILED DESCRIPTION OF THE INVENTION

After extensive and in-depth research, the inventor has developed for the first time that a cell-free fat extract shows excellent therapeutic effects on ovarian insufficiency and/or complications thereof (especially those caused by chemical damage).

### Terms

Unless otherwise defined, all technical and scientific terms used herein have the same meaning as commonly understood by one of ordinary skill in the art to which this invention belongs.

As used herein, the terms “include,” “comprise” and “contain” are used interchangeably to include not only closed definitions, but also semi-closed, and open definitions. In other words, the term includes “consist of” and “substantially consist of”.

As used in the text, the term “IGF-1” is referred to as insulin-like growth factors-1.

As used in the text, the term “BDNF” is referred to as brain-derived neurotrophic factor (BDNF).

As used in the text, the term “GDNF” is referred to as glialcellline-derived neurotrophic factor.

As used in the text, the term “bFGF” is referred to as basic fibroblast growth factor.

As used in the text, the term “VEGF” is referred to as vascular endothelial growth factor.

As used in the text, the term “TGF-β1” is referred to as transforming growth factor-β1.

As used in the text, the term “HGF” is referred to as hepatocyte growth factor.

As used in the text, the term “PDGF” is referred to as platelet derived growth factor.

As used in the text, the term “EGF” is referred to as epidermal growth factor.

As used in the text, the term “NT-3” is referred to as neurotrophins-3.

As used in the text, the term “GH” is referred to as growth hormone.

As used in the text, the term “G-CSF” is referred to as granulocyte colony stimulating factor.

### Cell-Free Fat Extract (CEFFE) and Preparation Method Therefor

As used herein, the terms “cell-free fat extract of the present invention”, “extract of the present invention”, “fat extract of the present invention” and the like are used interchangeably to refer to an extract (or extract liquid) derived from fatty tissue prepared without adding any solutions, solvents, small molecules, chemical agents, and biological additives during the preparation of the fat extract (other than the rinsing step). A typical process for preparing the extract of the present invention is as described above in the second aspect of the present invention. In addition, it should be understood that although the extract of the present invention does not need to add any additives (or additive ingredients) during the preparation process, some or a small amount of safe substance (such as a small amount of water) that does not negatively or adversely affect the activity of the extract of the present invention can also be added.

The cell-free fat extract of the present invention can be derived from human fatty tissue, which is purified from nano-fat by removing oil and cell/extracellular matrix components after centrifugation of fat tissue, and is a cell-free liquid which is easy-to-prepare and rich in various growth factors.

In another preferred embodiment of the present invention, the cell-free fat extract is a cell-free fat extract liquid.

The cell-free fat extract described in the present invention may include multiple cytokines. Representatively, the cell-free fat extract comprises one or more of IGF-1, BDNF, GDNF, TGF-β, HGF, bFGF, VEGF, TGF-β1, PDGF, EGF, NT-3, GH, and G-CSF.

In another preferred embodiment, in the cell-free fat extract, the concentration of IGF-1 is 5000-3000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, and more preferably 170-190 pg/ml.

In another preferred embodiment, the weight ratio of IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, preferably 6-12:1, and most preferably 8-9.5:1.

In another preferred embodiment, the weight ratio of GDNF to VEGF is 2-20:1, preferably 4-15:1, preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β1 to VEGF is 1-20:1, preferably 1-15:1, more preferably 1-10:1, more preferably 2-8:1, and more preferably 4-6:1.

In another preferred embodiment, the weight ratio of HGF to VEGF is 1-20:1, preferably 1-15:1, more preferably 1-10:1, more preferably 2-8:1, and more preferably 4-55:1.

In another preferred embodiment, the weight ratio of PDGF to VEGF is 0.1-3:1, preferably 0.2-2:1, more preferably 0.4-1.5:1, and most preferably 0.7-1.2:1.

Preferably, the cell-free fat extract of the present invention is prepared by the method as described in the second aspect of the present invention.

Representatively, the cell-free fat extract of the present invention is prepared by the following method:
(1) providing an fatty tissue raw material, shredding the fatty tissue raw material, and rinsing (eg, with physiological saline), thereby obtaining a rinsed fatty tissue;
(2) centrifuging the rinsed fatty tissue to obtain a layered mixture;
(3) removing the oil layer on top and the water layer at the bottom from the layered mixture and collecting the intermediate layer (that is, the fat layer containing fat cells);
(4) subjecting the intermediate layer to emulsification to obtain a emulsified fat mixture (also called nano-fat);
(5) subjecting the emulsified fat mixture to centrifugation to obtain a intermediate liquid layer, which is a fat primary extract; and
(6) subjecting the fat primary extract to filtration and sterilization to obtain the cell-free fat extract.

In another preferred embodiment, the cell-free fat extract is as described in the first aspect of the present invention.

In another preferred embodiment, in step (2), the centrifugation is performed at 800-2500 g, preferably 800-2000 g, more preferably 1000-1500 g, and most preferably 1100-1300 g.

In another preferred embodiment, in step (2), the centrifugation time is 1-15 minutes, preferably 1-10 minutes, more preferably 1-8 minutes, and most preferably 1-5 minutes.

In another preferred embodiment, in step (4), the emulsification is mechanical emulsification.

In another preferred embodiment, the mechanical emulsification is performed by repeatedly blowing through a syringe for many times (e.g. 20-200 times, preferably 20-150 times, preferably 20-100 times, preferably 30-50 times).

In another preferred embodiment, the blowing method is to repeatedly and uniformly push back through two 10 ml injection syringes connected to a three-way tube.

In another preferred embodiment, in step (4), the emulsification is a method of breaking up by a tissue homogenizer.

In another preferred embodiment, step (5) further comprises subjecting the emulsified fat mixture to freezing and thawing treatment before the emulsified fat mixture is subjected to centrifugation.

In another preferred embodiment, after freezing and thawing treatment, the thawed mixture is used for centrifugation.

In another preferred embodiment, the freezing temperature is −50° C. to −120° C., preferably −60° C. to −100° C., and more preferably −70° C. to −90° C.

In another preferred embodiment, the thawing temperature is 20-40° C., preferably 25-40° C., and more preferably 37° C.

In another preferred embodiment, the number of cycles for freezing and thawing is 1-5 (preferably 1, 2, 3, or 4).

In another preferred embodiment, in step (5), after centrifugation, the emulsified fat mixture is layered into four layers: the first layer is an oil layer, the second layer is a residual fat tissue layer, the third layer is a liquid layer (i.e. the intermediate liquid layer), and the fourth layer is a cell/tissue debris precipitation layer.

In another preferred embodiment, in step (5), the centrifugation is performed at 800-2500 g, preferably 800-2000 g, more preferably 1000-1500 g, and most preferably 1100-1300 g.

In another preferred embodiment, in step (5), the centrifugation time is 1-15 minutes, preferably 1-10 minutes, more preferably 2-8 minutes, and most preferably 3-7 minutes.

In another preferred embodiment, in the step (5), the first layer, the second layer, the third layer and the fourth layer are arranged sequentially from top to bottom.

In another preferred embodiment, in step (5), the intermediate liquid layer is a transparent or substantially transparent layer.

In another preferred embodiment, in step (6), the filtration is capable of removing fat cells from the fat primary extract.

In another preferred embodiment, in step (6), the filtration and sterilization are carried out through a filter (such as a 0.22 μm microporous filter membrane).

In another preferred embodiment, the filter is a microporous membrane filter.

In another preferred embodiment, the pore size of the microporous filter membrane is 0.05-0.8 μm, preferably 0.1-0.5 μm, more preferably 0.1-0.4 μm, more preferably 0.15-0.3 μm, more preferably 0.2-0.25 μm, and most preferably 0.22 μm.

In another preferred embodiment, in step (6), the filtration and sterilization are carried out by first filtering through a first filter that can filter cells, and then through a second filter (such as a 0.22 μm filter) that can filter pathogens (such as bacteria).

In another preferred embodiment, step (6) further comprises subpackaging the fat extract to form a subpackaged product. (The extract after subpackaging may be stored at −20° C. for later use; may be thawed at low temperature (e.g. −4° C.) or at room temperature then used directly, or thawed and stored at a low temperature (e.g. 4° C.) for a period of time before use).

### Use

The present invention provides a use of a cell-free fat extract for preparing a composition or a preparation, the composition or the preparation is used for preventing and/or treating ovarian insufficiency and/or complications thereof.

In the present invention, the term “prevent” means a method for preventing the onset of a disease and/or its accompanying symptoms or protecting a subject from developing the disease. The “prevent” used herein also includes delaying the onset of the disease and/or its accompanying symptoms and reducing the risk of getting sick in the subject.

The “treat” described in the present invention includes delaying and terminating the progression of the disease, or eliminating the disease, and does not require 100% inhibition, elimination and reversal. In some embodiments, the cell-free fat extract of the present invention reduces, inhibits, and/or reverses ovarian insufficiency, for example, by at least about 10%, by at least about 30%, by at least about 50%, or by at least about 80%, compared to the absence of the cell-free fat extract as described in the present invention.

The present invention also provides a method for preventing and/or treating ovarian insufficiency and/or complications thereof, comprising administering the cell-free fat extract as described in the present invention to a subject in need thereof.

In another preferred embodiment, the subject is a human or a non-human mammal.

In another preferred embodiment, the non-human mammal comprises a rodent, such as a rat, a mouse.

In another preferred embodiment, the mode of administration is oral, topical, or injection administration.

### Compositions and Administration

The compositions as described in the present invention include, but are not limited to, pharmaceutical compositions, food compositions, health care compositions, dietary supplements, and the like.

Representatively, the cell-free fat extract of the present invention can be prepared as pharmaceutical compositions in dosage forms such as tablets, capsules, powders, microgranules, solutions, lozenges, jellies, creams, spiritus, suspensions, tinctures, mud dressings, liniment, lotions, and aerosols, etc. Pharmaceutical compositions can be prepared by commonly known preparation techniques, and suitable pharmaceutical additives can be added to the drug.

The compositions of the present invention can also include pharmaceutically, food, health care product or dietary acceptable carriers. “Pharmaceutically, food, health care product or dietary acceptable carriers” means one or more compatible solid or liquid filler or gel substances that are suitable for human use and must have sufficient purity and sufficiently low toxicity. “Compatibility” herein refers to the ability of components of a composition to blend with the compounds of the invention and with each other without significantly reducing the efficacy of the compounds. Examples of pharmaceutically, food, health care product or dietary acceptable carriers include cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oil (such as soybean oil, sesame oil, peanut oil, olive oil, etc.), polyols (such as propylene glycol, glycerin, mannitol, sorbitol, etc.), emulsifier (such as Tween®), wetting agents (such as sodium dodecyl sulfate), colorants, flavoring agents, stabilizers, antioxidants, preservatives, pyrogen-free water, etc.

The administration methods of the compositions of the present invention are not particularly limited, and representative modes of administration include (but are not limited to) oral, parenteral (intravenous, intramuscular), topical administration, preferably oral administration and injection administration.

The dosage form of the composition or the preparation described in the present invention is an oral preparation, a topical preparation or an injection preparation. Representatively, solid dosage forms for oral administration include capsules, tablets, pills, powders and granules. In these solid dosage forms, the active compound is mixed with at least one conventional inert excipient (or carrier), such as sodium citrate or dicalcium phosphate, or mixed with the following components: (a) fillers or compatibilizers, e.g., starch, lactose, sucrose, glucose, mannitol and silicic acid; (b) binders, e.g., hydroxymethylcellulose, alginate, gelatin, polyvinylpyrrolidone, sucrose, and gum arabic; (c) humectants, e.g., glycerol; (d) disintegrants, e.g., agar, calcium carbonate, potato starch or tapioca starch, alginic acid, certain composite silicates, and sodium carbonate; (e) dissolution-retarding agents, e.g., paraffin; (f) absorption accelerators, e.g., quaternary amine compounds; (g) wetting agents, e.g., cetyl alcohol and glycerol monostearate; (h) sorbents, e.g., kaolin; and (i) lubricants, e.g., talc, calcium stearate, magnesium stearate, solid polyethylene glycol, sodium dodecyl sulfate, and the mixtures thereof. In capsules, tablets and pills, dosage forms may also contain buffers.

Solid dosage forms such as tablets, sugar pills, capsules, pills and granules may be prepared using coating and shell materials such as casing and other materials well known in the art. They can contain opaque agents.

Liquid dosage forms for oral administration include pharmaceutically acceptable emulsions, solutions, suspensions, syrups or tinctures. In addition to the active compounds, the liquid dosage forms may contain inert diluents conventionally used in the art such as water or other solvents, solubilizers and emulsifiers such as ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil, or the mixtures thereof and the like.

In addition to these inert diluents, the compositions may also contain auxiliaries such as wetting agents, emulsifiers, suspending agents, sweeteners, flavoring agents and spices.

In addition to the active ingredient, the suspensions may comprise suspending agents, such as ethoxylated isooctadecanol, polyoxyethylene sorbitol and dehydrated sorbitol esters, microcrystalline cellulose, methanolic aluminum, agar, and the mixtures thereof.

The compositions for parenteral injection may comprise physiologically acceptable sterile aqueous or anhydrous solutions, dispersions, suspensions or emulsions, and sterile powders for redissolution into sterile injectable solutions or dispersions. Suitable aqueous and non-aqueous carriers, diluents, solvents, or excipients include water, ethanol, polyols, and suitable mixtures thereof.

Dosage forms of the compounds of the present invention for topical administration include ointments, powders, patches, sprays and inhalants. The active ingredient is mixed with physiologically acceptable carriers and any preservatives, buffers or propellants that may be required if necessary under sterile conditions.

The cell-free fat extract of the present invention can be administered alone, or in combination with other drugs for preventing and/or treating ovarian insufficiency and/or complications thereof.

When the composition is administered, a safe and effective amount of the cell-free fat extract of the present invention is administrated to a human or a non-human animal (e.g., a rat, a mouse, a dog, a cat, a cow, a chicken, a duck, etc.) in need of treatment, wherein the dose when administered is an effective administration dosage which is pharmaceutically, food, nutraceutical or dietary acceptable. As used herein, the term “safe and effective amount” refers to an amount that produces function or activity to humans and/or animals and is acceptable to humans and/or animals. Those ordinary skilled in the art will understand that the “safe and effective amount” may vary depending on the form of the pharmaceutical composition, the route of administration, the excipient of the drug used, the severity of the disease, and the combination with other drugs. For example, for a person with a body weight of 60 kg, the daily dose is usually 0.1 to 1000 mg, preferably 1 to 600 mg, more preferably 2 to 300 mg. Of course, the specific dosage should also consider the route of administration, the patient's health state and other factors, which are all within the scope of skilled physician skills.

### The Main Advantages of the Present Invention Include:

1. The present invention for the first time discovered that cell-free fat extract has excellent preventive and therapeutic effects on ovarian insufficiency and/or complications thereof.
2. The cell-free fat extract described in the present invention is a cell-free component that can avoid cell related issues in clinical applications, such as genetic stability after cell processing, activity and survival rate of cell after injection, storage of cells after multiple administration, and immunogenicity of cells when using allogeneic fat. The cell-free fat extract of the present invention has the advantage of high safety and low side effect in preventing and treating ovarian insufficiency and/or complications thereof.

The present invention will be further explained below in combination with specific examples. It should be understood that these examples are only used to illustrate the present invention and not to limit the scope of the present invention. In the following examples, the test methods without specific conditions are usually in accordance with conventional conditions or the conditions recommended by the manufacturer. Unless otherwise specified, percentages and parts are calculated by weight.

### Example 1

### 1. Preparation of Cell-Free Fat Extract (CEFFE)

Fat is obtained by volunteers with informed consent. The preparation method of cell-free fat tissue extract liquid is as follows:
(1) Fat tissue was obtained from 6 healthy women who underwent conventional liposuction, with an average age of 31 years (24-36 years) old. After anesthesia with local injection of swelling solution, a 3 mm liposuction aspiration cannula with a large lateral hole (2 mm×7 mm) connected to a 20 mL syringe was used, and radial aspiration under negative pressure was manually performed to obtain fat, and the obtained fat was stood upright, and after removing of the swelling solution, it was rinsed 3 times with physiological saline.
(2) The rinsed fatty tissue was taken, placed in a centrifuge tube, then placed in a centrifuge and centrifuged at 1200 g at 4° C. for 3 minutes to obtain a layered mixture.
(3) For the layered mixture, the oil layer on top and the water layer at the bottom were removed and the intermediate layer (i.e. the fat layer containing fat cells) was collected.
(4) For the intermediate layer, two 10 ml syringes connected to a three-way tube were pushed repeatedly and uniformly for 30 times, thus performing mechanical emulsification and obtaining a mechanically emulsified fat mixture (also called nano-fat).
(5) The mechanically emulsified fat mixture was placed into a −80° C. refrigerator for freezing, and then thawed in a 37° C. water bath, and after a single freeze-thaw cycle, the thawed fat mixture was centrifuged at 1200 g at 4° C. for 5 minutes to obtain a layered mixture, which was divided into 4 layers, the first layer is an oil layer, the second layer is a residual fat tissue layer, the third layer is a liquid layer, and the fourth layer is a cell/tissue debris precipitation layer, the oil layer and the residual fat tissue layer were removed and the liquid layer was aspirated, avoiding contamination of the cell/tissue debris precipitation layer during the aspiration process, thereby obtaining fat primary extract.
(6) The obtained fat primary extract was filtered and sterilized through a 0.22 μm filter, thereby sterilizing and removing any live cells that may be mixed, thereby obtaining the cell-free fat extract (CEFFE) that was subpackaged and stored frozen at −20° C. and thawed at 4° C. when used.

For the cell-free fat extract liquid prepared, the content of cytokines, including IGF-1, BDNF, GDNF, bFGF, VEGF, TGF-β1, HGF and PDGF, was detected by ELISA immunosorbent assay kit. The average concentrations of 6 samples were as follows: IGF-1 (9840.6 pg/ml), BDNF (1764.5 pg/ml), GDNF (1831.9 pg/ml), bFGF (242.3 pg/ml), VEGF (202.9 pg/ml), TGF-β1 (954.5 pg/ml), HGF (898.4 pg/ml), and PDGF (179.9 pg/ml).

### 2. Experimental Methods and Results

The therapeutic effect of cell-free fat extract (CEFFE) on ovarian insufficiency and complications thereof caused by chemical damage was investigated. Specifically, a mouse model of ovarian insufficiency (POI) caused by chemical drugs was established, CEFFE was applied for treatment, and the degree of improvement in mouse ovarian function was observed through various indicators.

The POI group selected C57 mice for model establishment, with intraperitoneal injection of a loading dose of 120 mg/kg of cyclophosphamide by one-time combined with intradermal injection of 12 mg/kg of busulfan in a total of 0.1 mL (PBS was used as the solvent). The control group was subjected to experimental observation using normal mice without injection of chemotherapeutic drugs as the subject.

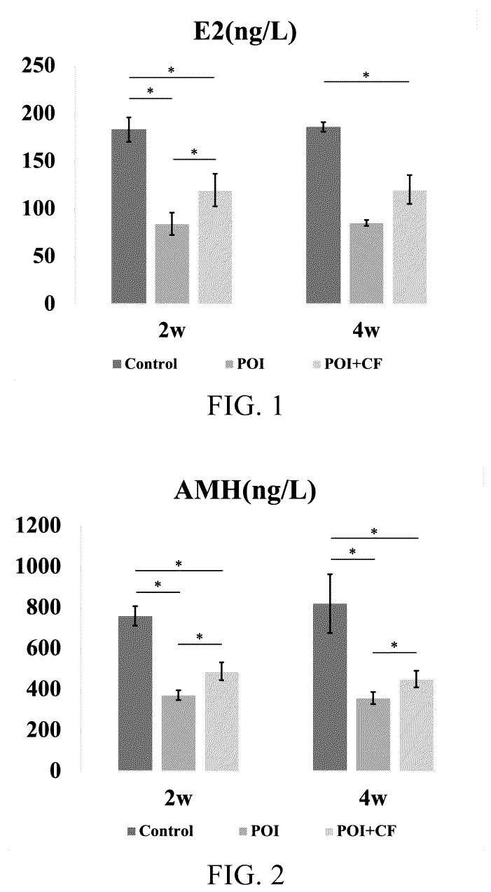

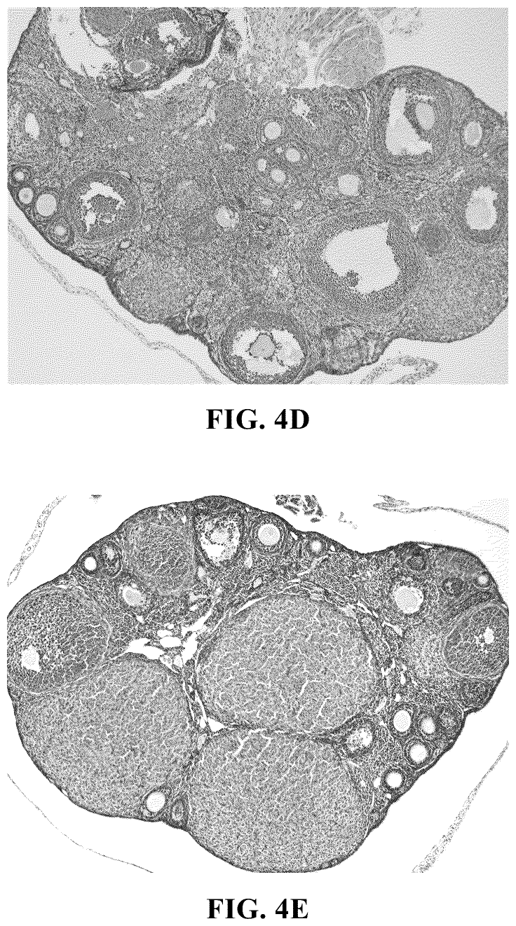

In vivo experiments: After establishment of a successful POI mouse model was confirmed, CEFFE treatment was performed on mice. The experimental group (POI+CF) selected CEFFE preparations with a protein content of above 3.0 for intraperitoneal injection, 0.1 mL/day, and continuous treatment for 7 days. The ovaries of part of the mice were sampled at 2 and 4 weeks after treatment for testing of sex hormone levels and ovarian histology. The results of sex hormone levels are shown in FIGS. 1, 2, and 3, and the ovaries histological results are shown in FIG. 4.

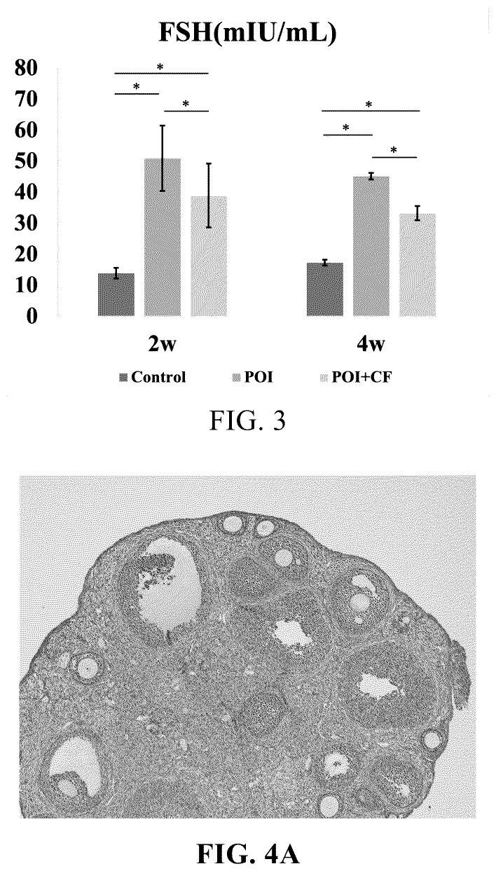

FIGS. 1 and 2 illustrate that the levels of E2 (estradiol) and AMH (anti-Müllerian hormone) significantly decreased in mice with ovarian insufficiency caused by chemotherapy. After intraperitoneal injection of CEFFE, E2 hormone levels significantly increased and remained stable until the four weeks end point of observation time. The difference in hormone levels before and after CEFFE injection is statistically significant. The results demonstrate that CEFFE has significant enhancing effects on E2 hormone levels and AMH hormone levels in chemotherapy induced ovarian insufficiency mice, which is beneficial for the recovery of ovarian function. (*P<0.05)

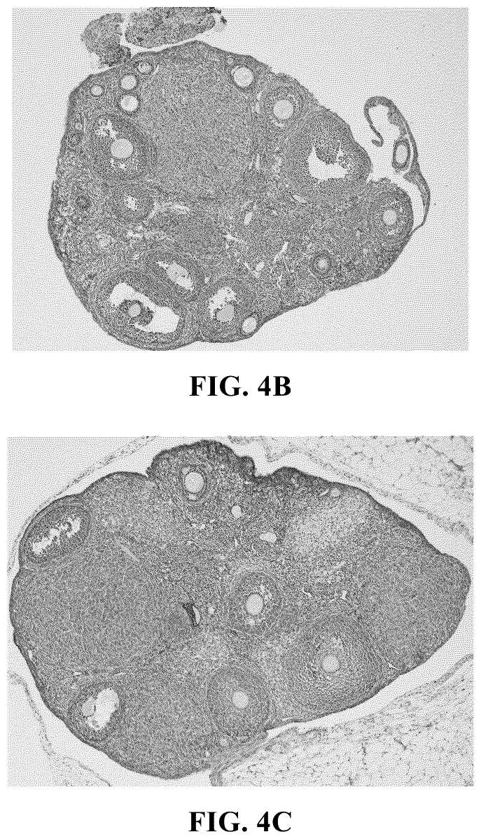

FIG. 3 illustrates that chemotherapy induced a significant increase in FSH (follicle stimulating hormone) hormone levels in mice with ovarian insufficiency. After intraperitoneal injection of CEFFE, the FSH values showed a significant decrease, and the value continued to decrease and gradually stabilized at the four week observation endpoint. The difference in hormone levels before and after CEFFE injection is statistically significant. This result demonstrates that CEFFE has a significant reducing effect on FSH hormone levels in chemotherapy induced ovarian insufficiency mice, which is beneficial for the recovery of ovarian function (*P<0.05).

In the POI 2w group, it was seen that a large number of secondary follicles regressed and atretic, with a small amount of inflammatory cell infiltration (FIG. 4A). In the POI+CF 2w group, the follicle structure was found to be basically normal, and the medulla part collapsed (FIG. 4B). In the POI 4w group, a decrease in the number of primordial follicles, corpus luteum, and more interstitial glands were observed, indicating that ovulation had just occurred (FIG. 4C). A small amount of inflammatory cell infiltration were observed and the overall structure was normal in the POI+CF group after 4 week (FIG. 4D). Normal ovarian slices were shown in FIG. 4E.

It was found that CEFFE can promote the recovery of ovarian function and structure of ovarian insufficiency caused by chemical damage.

Therefore, CEFFE has excellent therapeutic effects on ovarian insufficiency and complications thereof caused by chemical damage.

All documents mentioned herein are incorporated by reference in this application as if each document were individually incorporated by reference. In addition, it should be understood that after reading the above teaching content of the present invention, those skilled in the art can make various changes or modifications to the present invention, and these equivalent forms also fall within the scope defined by the appended claims of the present application.

1-15. (canceled)

16. A method for preventing and/or treating ovarian insufficiency and/or complications thereof, comprising administering (i) a cell-free fat extract or (ii) a composition or preparation containing the cell-free fat extract to a subject in need thereof.

17. The method according to claim 16, wherein the complications of ovarian insufficiency are selected from the group consisting of secondary amenorrhea, decreased estrogen level, increased gonadotropin level, decreased number of follicles, decreased follicle quality, premature amenorrhea, infertility, and the combinations thereof.

18. The method according to claim 16, wherein the ovarian insufficiency includes ovarian insufficiency caused by chemical damage.

19. The method according to claim 18, wherein the chemical damage includes damage caused by chemotherapeutic drugs.

20. The method according to claim 19, wherein the chemotherapeutic drugs are selected from the group consisting of cyclophosphamide, busulfan, paclitaxel, doxorubicin, and the combinations thereof.

21. The method according to claim 16, wherein the preventing and/or treating ovarian insufficiency and/or complications thereof include:
(i) promoting the recovery of ovarian function;
(ii) enhancing fertility.

22. The method according to claim 21, wherein the promoting the recovery of ovarian function includes one or more features selected from the group consisting of:
(i) increasing estradiol hormone level;
(ii) increasing anti-Müllerian hormone level;
(iii) decreasing follicle stimulating hormone level;
(iv) promoting the restore of follicular structure.

23. The method according to claim 16, wherein the composition or the preparation comprises a pharmaceutical composition or preparation, a food composition or preparation, a health care composition or preparation, or a dietary supplement.

24. The method according to claim 16, wherein the dosage form of the composition or the preparation is an oral preparation, a topical preparation, or an injectable preparation.

25. The method according to claim 16, wherein the mode of administration is oral, topical, or injection administration.

26. The method according to claim 16, wherein the cell-free fat extract contains no cells and contains no lipid droplets.

27. The method according to claim 26, wherein “contains no lipid droplets” means that in the cell-free fat extract, the volume of oil droplets accounts for less than 1% of the total liquid, preferably less than 0.5%, more preferably less than 0.1%;
the “cell-free” means that the average number of cells in 1 ml of the cell-free fat extract is ≤1, preferably ≤0.5, more preferably ≤0.1, or 0.

28. The method according to claim 16, wherein the cell-free fat extract is a naturally obtained nanofat extract without additive.

29. The use according to claim 16, wherein the cell-free fat extract is obtained by subjecting an fatty tissue to emulsification and then centrifugation.

30. The use according to claim 16, wherein the cell-free fat extract is prepared by the following method:
(1) providing an fatty tissue raw material, shredding the fatty tissue raw material, and rinsing (eg, with physiological saline), thereby obtaining a rinsed fatty tissue;
(2) centrifuging the rinsed fatty tissue to obtain a layered mixture;
(3) removing the oil layer on top and the water layer at the bottom from the layered mixture and collecting the intermediate layer (that is, the fat layer containing fat cells);
(4) subjecting the intermediate layer to emulsification to obtain a emulsified fat mixture (also called nano-fat);
(5) subjecting the emulsified fat mixture to centrifugation to obtain a intermediate liquid layer, which is a fat primary extract; and
(6) subjecting the fat primary extract to filtration and sterilization to obtain the cell-free fat extract.

31. The use according to claim 30, wherein in step (5), after centrifugation, the emulsified fat mixture is layered into four layers: the first layer is an oil layer, the second layer is a residual fat tissue layer, the third layer is a liquid layer (i.e. the intermediate liquid layer), and the fourth layer is a cell/tissue debris precipitation layer.

32. The use according to claim 30, wherein in step (4), the emulsification is mechanical emulsification,
the mechanical emulsification is performed by repeatedly blowing through a syringe for many times (e.g. 20-200 times, preferably 20-150 times, preferably 20-100 times, preferably 30-50 times).
